(19) United States
(12) Patent Application Publication
(10) Pub. No.: US 2017/0334865 A1
(43) Pub. Date: Nov. 23, 2017

(54) DDR2 INHIBITORS AND METHODS OF USING

(71) Applicants: Whitney GRITHER, St. Louis, MO (US); WASHINGTON UNIVERSITY, St. Louis, MO (US)

(72) Inventors: Whitney Grither, St. Louis, MO (US); Gregory Longmore, St. Louis, MO (US)

(73) Assignee: Washington University, St. Louis, MO (US)

(21) Appl. No.: 15/522,201

(22) PCT Filed: Oct. 30, 2015
(86) PCT No.: PCT/US15/58283

§ 371 Date: Apr. 26, 2017

Related U.S. Application Data
(60) Provisional application No. 62/073,194, filed on Oct. 31, 2014.

(51) Int. Cl. C07D 265/38; A61K 31/538; A61K 45/06; C07D 413/12

(52) U.S. Cl. CPC C07D 265/38; C07D 413/12; A61K 31/538; A61K 45/06

(57) ABSTRACT

Compounds that inhibit activation of Discoidin Domain Receptor 2 (DDR2) and methods of using the compounds to inhibit DDR2 activation, inhibit migration of cells expressing DDR2, and treat diseases or disorders associated with DDR2 dysfunction.

### CROSS REFERENCE TO RELATED APPLICATIONS

This application claims the benefit of U.S. provisional application No. 62/073,194, filed Oct. 31, 2014, which is hereby incorporated by reference in its entirety.

### FIELD OF THE INVENTION

The present disclosure relates to compounds and methods for treating diseases or disorders associated with DDR2 dysfunction.

### BACKGROUND

Discoidin Domain Receptor 2 (DDR2) is a receptor tyrosine kinase (RTK) that utilizes the extracellular matrix protein collagen as its ligand. In addition to its kinase function, DDR2 also functions to promote cellular adhesion through activation of β1-integrins. DDR2 is highly expressed at the leading edge of many invasive breast tumor samples. It is believed to be critical for metastasis of many cancer cells such as breast cancer cells. DDR2 is also considered to be an important target for other cancers (e.g., ovarian, lung, head and neck, pancreatic), inflammation and arthritis (e.g., osteoarthritis, rheumatoid arthritis), and fibrosis (e.g., lung, liver, kidney, skin).

Most clinically available drugs targeting RTKs are small molecule inhibitors of the intracellular tyrosine kinase (TK) activity, such as imatinib, nilotinib, or dasatinib, or humanized antibodies directed against the extracellular ligand-binding site or receptor dimerization sites. Formation of active RTK signaling complexes also involves conformational changes in the extracellular domain that position the intracellular TK domains for signal transduction.

Small molecule inhibitors have been developed to target DDR2 (e.g., dasatinib, WO 2005092896, WO 2014032755). These compounds inhibit kinase activity by blocking binding of ATP in the active pocket of DDR2. However, such targeting can be non-selective for DDR2, and off target effects make such kinase inhibitors sub-optimal for treatment.

Alternative compounds and methods for inhibiting DDR2 activity and treating cancers and/or inflammatory disorders are needed.

### SUMMARY

Among the various aspects of the present disclosure encompasses a compound of Formula (I):

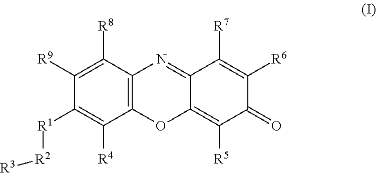

wherein:
R¹ is O or NH;
R² is alkyl, substituted alkyl, carbonyl, ester, amide, sulfinyl, or sulfonyl;
R³ is aryl, substituted aryl, heterocyclic, or substituted heterocyclic;
R⁴, R⁵, R⁶, R⁷, R⁸, and R⁹ independently are hydrogen, OR²⁰, NR²⁰R²¹, alkyl, substituted alkyl, halo, or thiol; and
R²⁰ and R²¹ independently are hydrogen, hydrocarbyl, or substituted hydrocarbyl;
provided that when R⁴-R⁹ are hydrogen, R¹ is O, and R² is methylene, then R³ is other than phenyl, hydroxyphenyl, hydrocarbyloxyphenyl, substituted hydrocarbyloxyphenyl, halophenyl, or haloalkylphenyl, or when R⁴-R⁹ are hydrogen, R¹ is O, and R² is carbonyl, then R³ is other than phenyl, hydroxyphenyl, methoxyphenyl, or furyl.

Another aspect of the present disclosure encompasses a method for inhibiting DDR2 activation. The method comprises contacting DDR2 with an effective amount of a compound of Formula (I):

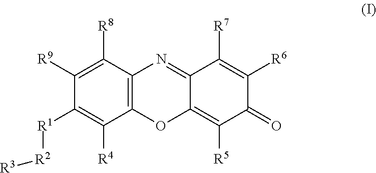

wherein:
R¹ is O or NH;
R² is alkyl, substituted alkyl, carbonyl, ester, amide, sulfinyl, sulfinyl, or is absent;
R³ is hydrogen, hydrocarbyl, or substituted hydrocarbyl;
R⁴, R⁵, R⁶, R⁷, R⁸, and R⁹ independently are hydrogen, OR²⁰, NR²⁰R²¹, alkyl, substituted alkyl, halo, or thiol; and
R²⁰ and R²¹ independently are hydrogen, hydrocarbyl, or substituted hydrocarbyl.

Yet another aspect of the present disclosure provides a method for inhibiting cell migration. The method comprises contacting a cell that expresses DDR2 with an effective amount of a compound of Formula (I):

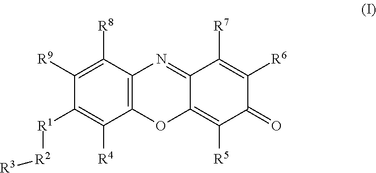

wherein:
R¹ is O or NH;
R² is alkyl, substituted alkyl, carbonyl, ester, amide, sulfinyl, sulfinyl, or is absent;
R³ is hydrogen, hydrocarbyl, or substituted hydrocarbyl;
R⁴, R⁵, R⁶, R⁷, R⁸, and R⁹ independently are hydrogen, OR²⁰, NR²⁰R²¹, alkyl, substituted alkyl, halo, or thiol; and
R²⁰ and R²¹ independently are hydrogen, hydrocarbyl, or substituted hydrocarbyl.

Still another aspect of the present disclosure encompasses a method for treating a disease or disorder associated with DDR2 dysfunction. The method comprises administering to a subject in need thereof an effective amount of a compound of Formula (I):

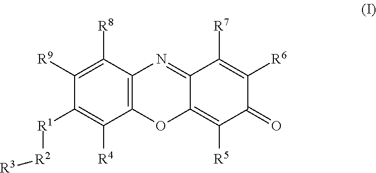

wherein:
R¹ is O or NH;
R² is alkyl, substituted alkyl, carbonyl, ester, amide, sulfinyl, sulfinyl, or is absent;
R³ is hydrogen, hydrocarbyl, or substituted hydrocarbyl;
R⁴, R⁵, R⁶, R⁷, R⁸, and R⁹ independently are hydrogen, OR²⁰, NR²⁰R²¹, alkyl, substituted alkyl, halo, or thiol; and
R²⁰ and R²¹ independently are hydrogen, hydrocarbyl, or substituted hydrocarbyl.

Other aspects and iterations of the disclosure are described in more detail below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A shows DDR2 binding activity (absorbance at 490 nm) in the presence of increasing concentration of substrate in control (DMSO) and three concentrations of WRG-11.

FIG. 1B presents DDR2 binding activity (absorbance at 490 nm) in the presence of increasing concentration of substrate in control (DMSO) and three concentrations of WRG-28.

FIG. 1C shows DDR2 resolved in the absence or presence of WRG-11 on a polyacrylamide gel under non-reducing conditions. Dimers of DDR2 run at 150 kDa.

FIG. 1D shows that binding of WRG-R11 does not irreversibly modify the ability of DDR2 to bind collagen.

FIG. 1E shows that DDR2 inhibitors inhibit the binding of DDR2 to collagen, but do not affect the collagen binding ability of DDR1 or α1β1 integrin. Data are means±SEM.

FIG. 2A presents Western blots of DDR2 probed with anti-DDR2 or anti-pY antibodies. HEK293 cells were incubated in the presence or absence of collagen and the indicated concentrations of WRG-11 or WRG-28. The ratio of phospho-DDR2 to DDR2 is presented below each blot.

FIG. 2B shows proliferation of control (SCR RNAi), DDR2 depleted (DDR2 RNAi), and inhibitor treated (SCR RNAi+WRG-11) 4T1 breast cancer cells. *P<0.01.

FIG. 2C presents the invasive index (relative photon flux) of the cells identified in FIG. 2B in an in vitro gel matrix cell invasion assay. *P<0.01.

FIG. 2D shows migration (i.e., percent wound closure) of mouse fibroblasts treated without or with WRG-11 in a wound healing assay. *P<0.01.

FIG. 2E presents migration (relative distance traveled) of control (shSCR), DDR2 depleted (shDDR2), and inhibitor treated (shSCR+WRG-28) BT549 human breast cancer cells through a 3D collagen matrix.

FIG. 3A illustrates impaired signaling downstream of DDR2. Shown are Western blots of proteins from untreated or HEK293 cells treated with WRG-11.

FIG. 3B shows impaired signaling downstream of DDR2 Shown are Western blots of proteins from untreated or HEK293 cells treated with WRG-28.

FIG. 4A illustrates in vivo studies with DDR2 inhibitors. Presented are representative images of 4T1-Snail-CBG labeled tumors in control or WRG-28 treated mice.

FIG. 4B plots the bioluminescence of 4T1-Snail-CBG labeled tumors in control or WRG-28 treated mice via the indicated administration routes.

FIG. 5A presents the percentage of GFP-tumor positive areas in the lungs of control (SCR), DDR2 depleted (DDR2), inhibitor treated (shSCR+WRG-28) mice. Two sections through each of five lobes per animal were quantified. Data derived from two experiments of four mice per condition. Means and SEM. **P<0.002.

FIG. 5B shows representative images of mice described in FIG. 5A at baseline and after 7 days.

FIG. 5C shows reduced metastatic lung tumors in DDR2 depleted (shDDR2) and inhibitor treated (shSCR+WRG-28) mice relative to control (shSCR) mice. 4T1-GFP-luc expressing cells were injected by tail vein. Means and SEM; data derived from one experiment of four mice per condition. ** P<0.01.

FIG. 6A plots the number of peritoneal cavity nodules in mice injected IP with control (shSCR) or DDR2 depleted (shDDR2) A2780 human ovarian cancer lines in an ovarian cancer metastasis assay.

FIG. 6B presents tumor weight in control (shSCR) and DDR2 depleted (shDDR2) A2780 mice in the ovarian cancer metastasis assay.

### DETAILED DESCRIPTION

The present disclosure provides phenoxazinone derivatives that inhibit DDR2 activation in an allosteric manner. Most clinically available drugs targeting RTKs are small molecule inhibitors of the intracellular tyrosine kinase (TK) activity or humanized antibodies directed against the extracellular ligand-binding site (orthosteric inhibitors) or receptor dimerization sites. Formation of active RTK signaling complexes also involves conformational changes in the extracellular domain that position the intracellular TK domains for signal transduction. Therefore, allosteric modulation of RTK activation offers new and significant therapeutic advantages over traditional orthosteric drugs, including greater safety (reduced toxicity) and selectivity. Also provided herein are compositions comprising said compounds and methods of using said compounds to inhibit DDR2 activation, inhibit migration and/or invasion of cells that express DDR2, and treat diseases or disorder associated with DDR2 dysfunction.

### (I) Compounds of Formula (I)

One aspect of the present disclosure encompasses compounds of Formula (I):

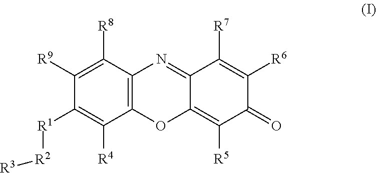

wherein:
R¹ is O or NH;
R² is alkyl, substituted alkyl, carbonyl, ester, amide, sulfinyl, or sulfonyl;
R³ is aryl, substituted aryl, heterocyclic, or substituted heterocyclic;
R⁴, R⁵, R⁶, R⁷, R⁸, and R⁹ independently are hydrogen, OR²⁰, NR²⁰R²¹, alkyl, substituted alkyl, halo, or thiol; and
R²⁰ and R²¹ independently are hydrogen, hydrocarbyl, or substituted hydrocarbyl;
provided that when R⁴-R⁹ are hydrogen, R¹ is O, and R² is methylene, then R³ is other than phenyl, hydroxyphenyl, hydrocarbyloxyphenyl, substituted hydrocarbyloxyphenyl, halophenyl, or haloalkylphenyl, or when R⁴-R⁹ are hydrogen, R¹ is O, and R² is carbonyl, then R³ is other than phenyl, hydroxyphenyl, methoxyphenyl, or furyl.

In various embodiments, R² may be C₁-C₅ alkyl or C₁-C₅ substituted alkyl. For example, R² may be —CH₂— (methylene), —(CH₂)₂—, —(CH₂)₃—, F—(CH₂)₄—, or —(CH₂)₅—. Alternatively, R² may be an alkyl comprising one or more alkyl, amino, hydroxyl, keto, nitro, phospho, or thiol substituents. In other embodiments, R² may be carbonyl (—C(O)—), ester (—C(O)O—), amide (—C(O)NH—), sulfinyl (—S(O)—), or sulfonyl (—S(O)₂—).

As mentioned above, R³ may be aryl, substituted aryl, heterocyclic, or substituted heterocyclic. As used herein, “heterocyclic” refers to saturated or unsaturated, monocyclic or bicyclic, aromatic or non-aromatic groups having at least one heteroatom in at least one ring, and “aryl” refers to homocyclic (i.e., carbocyclic) aromatic groups. In some embodiments, R³ may be phenyl, substituted phenyl, naphthyl, substituted naphthyl, furyl, substituted furyl, benzofuryl, substituted benzofuryl, oxazolyl, substituted oxazolyl, isoxazolyl, substituted isoxazolyl, oxadiazolyl, substituted oxadiazolyl, benzoxazolyl, substituted benzoxazolyl, benzoxadiazolyl, substituted benzoxadiazolyl, pyrrolyl, substituted pyrrolyl, pyrazolyl, substituted pyrazolyl, imidazolyl, substituted imidazolyl, triazolyl, substituted triazolyl, tetrazolyl, substituted tetrazolyl, pyridyl, substituted pyridyl, pyrimidyl, substituted pyrimidyl, pyrazinyl, substituted pyrazinyl, pyridazinyl, substituted pyridazinyl, piperidyl, substituted piperidyl, indolyl, substituted indolyl, isoindolyl, substituted isoindolyl, indolizinyl, substituted indolizinyl, morpholyl, substituted morpholyl, benzimidazolyl, substituted benzimidazolyl, indazolyl, substituted indazolyl, benzotriazolyl, substituted benzotriazolyl, tetrazolopyridazinyl, substituted tetrazolopyridazinyl, carbazolyl, substituted carbazolyl, purinyl, substituted purinyl, quinolinyl, substituted quinolinyl, isoquinolinyl, substituted isoquinolinyl, imidazopyridyl, or substituted imidazopyridyl. In specific embodiments, R³ may be phenyl, substituted phenyl (e.g., phenylamine, hydroxyphenyl, methoxyphenyl, dihalomethoxyphenyl, benzoate, methyl benzoate, ethyl benzoate, benyzl, substituted benzyl, phenylsulfinyl, phenylsulfonyl, etc.), pyridyl, substituted pyridyl, furyl, or substituted furyl. In certain embodiments, R³ may be sulfonyl substituted phenyl (e.g., N-methylbenzenesulfonamide, N-ethylbenzenesulfonamide, or phenylsulfonylmorpholine).

In certain embodiments, R⁴, R⁵, R⁶, R⁷, R⁸, and R⁹ independently may be hydrogen, hydroxy, C₁-C₅ alkyoxy, amino, amine, C₁-C₅ alkyl, C₁-C₅ substituted alkyl, halo, or thio. In specific embodiments, R⁴, R⁵, R⁶, R⁷, R⁸, and R⁹ are hydrogen.

In embodiments in which R²⁰ and R²¹ are present, R²⁰ and R²¹ independently may be hydrogen, alkyl (wherein alkyl may be linear, branched, or cyclic), substituted alkyl, alkenyl, substituted alkenyl, alkynyl, substituted alkynyl, aryl, or substituted aryl. In some embodiments, R²⁰ and R²¹ independently may be hydrogen, C₁-C₆ alkyl, or C₁-C₆ substituted alkyl.

In embodiments in which R⁴-R⁹ are hydrogen, R¹ is O, and R² is methylene, then R³ is other than phenyl, hydroxyphenyl, hydrocarbyloxyphenyl, substituted hydrocarbyloxyphenyl, halophenyl, or haloalkylphenyl, and in embodiments in which R⁴-R⁹ are hydrogen, R¹ is O, and R² is carbonyl, then R³ is other than phenyl, hydroxyphenyl, methoxyphenyl, or furyl.

In specific embodiments, R¹ is O or NH, R² is methylene, carbonyl, amide, or sulfonyl, R³ is phenyl, substituted phenyl, pyridyl, substituted pyridyl, furyl, or substituted furyl, and R⁴-R⁹ are hydrogen.

Specific compounds of Formula (I) are presented below:

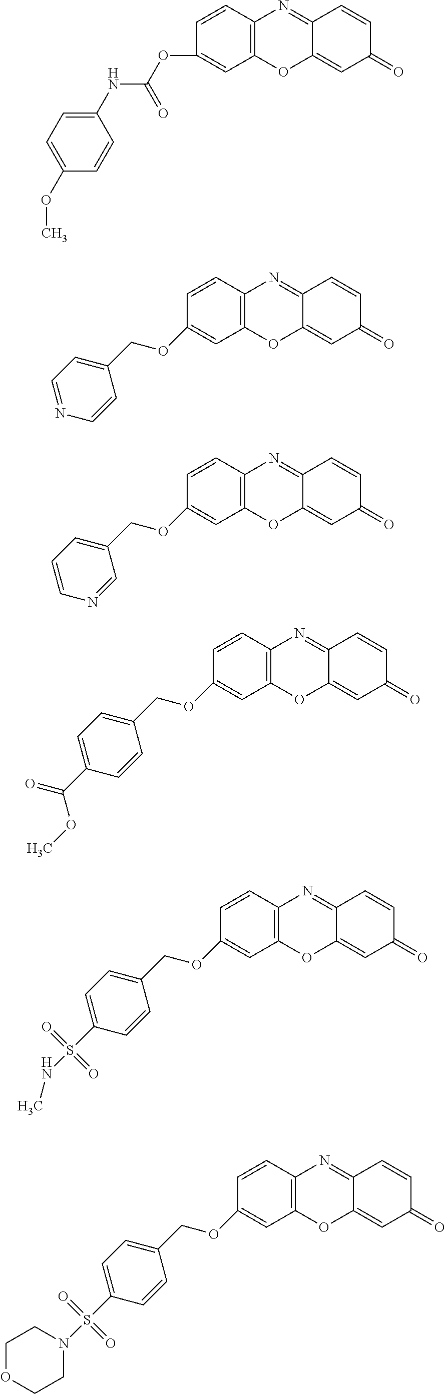 [SMILES: COc1ccc(NC(=O)Oc2ccc3nc4ccc(=O)cc-4oc3c2)cc1 ; O=c1ccc2nc3ccc(OCc4ccncc4)cc3oc-2c1 ; O=c1ccc2nc3ccc(OCc4cccnc4)cc3oc-2c1 ; COC(=O)c1ccc(COc2ccc3nc4ccc(=O)cc-4oc3c2)cc1 ; CNS(=O)(=O)c1ccc(COc2ccc3nc4ccc(=O)cc-4oc3c2)cc1 ; O=c1ccc2nc3ccc(OCc4ccc(S(=O)(=O)N5CCOCC5)cc4)cc3oc-2c1]

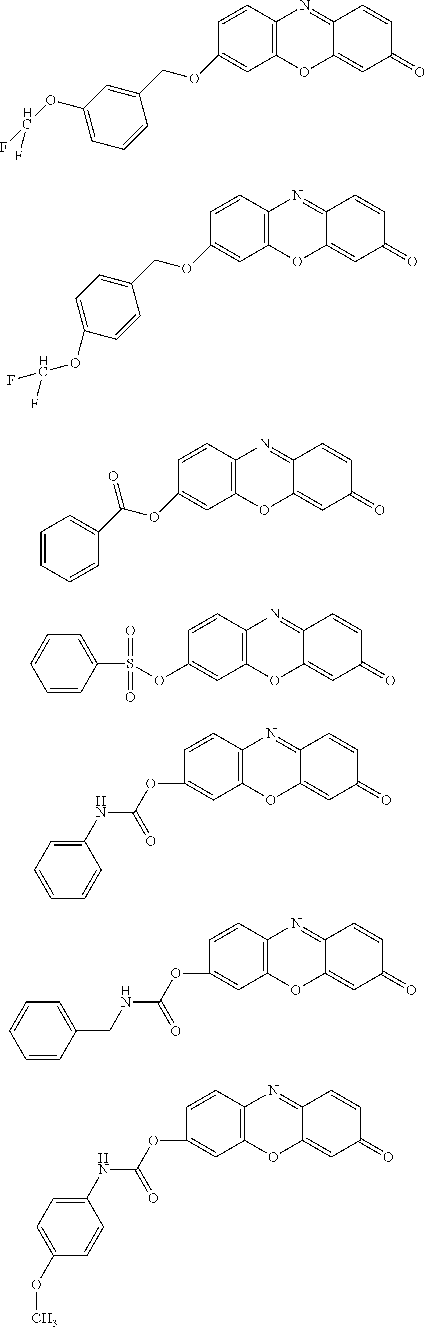

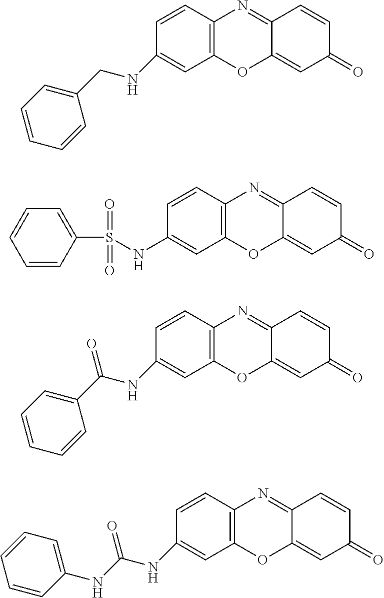 [SMILES: O=c1ccc2nc3ccc(NCc4ccccc4)cc3oc-2c1 ; O=c1ccc2nc3ccc(NS(=O)(=O)c4ccccc4)cc3oc-2c1 ; O=C(Nc1ccc2nc3ccc(=O)cc-3oc2c1)c1ccccc1 ; O=C(Nc1ccccc1)Nc1ccc2nc3ccc(=O)cc-3oc2c1]

Persons skilled in the art understand that the compounds of Formula (I) may be prepared by a variety of methods. Example 1 below describes several methods.

### (II) Compositions Comprising Compounds of Formula (I)

Another aspect of the present disclosure encompasses compositions comprising at least one compound of Formula (I) and at least one pharmaceutically acceptable excipient. The compositions disclosed herein may further comprise at least one additional active pharmaceutical ingredient (API). For example, the compositions may further comprise another DDR2 inhibitor, another receptor tyrosine kinase (RTK) inhibitor, a chemotherapeutic agent, and/or an inflammatory agent.

(a) Compounds of Formula (I)

The composition comprises at least one compound of Formula (I) or a pharmaceutically acceptable salt of the compound of Formula (I):

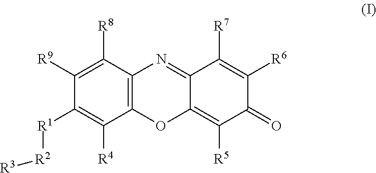

wherein R¹, R², R³, R⁴, R⁵, R⁶, R⁷, R⁸, and R⁹ are as defined above in Section (I).

The amount of the compound of Formula (I) included in the composition can and will vary depending upon the identity of the compound of Formula (I).

(b) Pharmaceutically Acceptable Excipients

The pharmaceutically acceptable excipient may be a diluent, a binder, a filler, a buffering agent, a pH modifying agent, a disintegrant, a dispersant, a preservative, a lubricant, taste-masking agent, a flavoring agent, a coloring agent, or combination thereof. The amount and types of excipients utilized to form pharmaceutical compositions may be selected according to known principles of pharmaceutical science.

In one embodiment, the excipient may comprise at least one diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

(c) Optional Additional APIs

In certain embodiments, the compositions may further comprise at least one additional DDR2 inhibitor. In general, the additional DDR2 inhibitor will be an orthosteric inhibitor. In one instance, the additional DDR2 inhibitor may be dasatinib (sold under the trade name SPYRCEL®). In other instances, the additional DDR2 inhibitor may be any of the compounds disclosed in WO 2005092896 or WO 2014032755, the disclosures of which are hereby incorporated by reference in their entireties.

In other embodiments, the compositions may further comprise at least one additional RTK inhibitor. Example of suitable RTK inhibitors include without limit afatinib, axitinib, bosutinib, cediranib, erlotinib, gefitinib, grandinin, imatinib, lapatinib, lestaurtinib, neratinib, nilotinib, pazopanib, quizartinib, regorafenib, semaxanib, sorafenib, sossunitinib, tivozanib, toceranib, vandetanib, and vatalanib.

In additional embodiments, the compositions may further comprise one or more chemotherapeutic agents. The chemotherapeutic agent may be an alkylating agent, an anti-metabolite, an anti-tumor antibiotic, an anti-cytoskeletal agent, a topoisomerase inhibitor, an anti-hormonal agent, a targeted therapeutic agent, or a combination thereof. Non-limiting examples of suitable alkylating agents include altretamine, benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lomustine (CCNU), mechlorethamine, mechlorethamine oxide hydrochloride, melphalan, meturedopa, nimustine, novembichin, phenesterine, piposulfan, prednimustine, ranimustine; temozolomide, thiotepa, triethylenemelamine, trietylenephosphoramide, triethylenethiophosphaoramide, trimethylolomelamine, trofosfamide, uracil mustard and uredopa. Suitable anti-metabolites include, but are not limited to aminopterin, ancitabine, azacitidine, 6-azauridine, capecitabine, carmofur (1-hexylcarbomoyl-5-fluorouracil), cladribine, cytarabine or cytosine arabinoside (Ara-C), dideoxyuridine, denopterin, doxifluridine, enocitabine, floxuridine, fludarabine, 5-fluorouracil, gemcetabine, hydroxyurea, leucovorin (folinic acid), 6-mercaptopurine, methotrexate, pemetrexed, pteropterin, thiamiprine, trimetrexate, and thioguanine. Non-limiting examples of suitable anti-tumor antibiotics include aclacinomysin, actinomycins, adriamycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mithramycin, mycophenolic acid, nogalamycin, olivomycins, peplomycin, plicamycin, potfiromycin, puromycin, quelamycin, rodorubicin, streptonigrin, streptozocin, tubercidin, valrubicin, ubenimex, zinostatin, and zorubicin. Non-limiting examples of suitable anti-cytoskeletal agents include colchicines, docetaxel, macromycin, paclitaxel, vinblastine, vincristine, vindesine, and vinorelbine. Suitable topoisomerase inhibitors include, but are not limited to, amsacrine, etoposide (VP-16), irinotecan, mitoxantrone, RFS 2000, teniposide, and topotecan. Non-limiting examples of suitable anti-hormonal agents such as aminoglutethimide, aromatase inhibiting 4(5)-imidazoles, bicalutamide, finasteride, flutamide, goserelin, 4-hydroxytamoxifen, keoxifene, leuprolide, LY117018, mitotane, nilutamide, onapristone, raloxifene, tamoxifen, toremifene, and trilostane. Examples of targeted therapeutic agents include, without limit, monoclonal antibodies such as alemtuzumab, epratuzumab, gemtuzumab, ibritumomab tiuxetan, rituximab, tositumomab, and trastuzumab; protein kinase inhibitors such as bevacizumab, cetuximab, dasatinib, erlotinib, gefitinib, imatinib, lapatinib, mubritinib, nilotinib, panitumumab, pazopanib, sorafenib, sunitinib, and vandetanib; angiogeneisis inhibitors such as angiostatin, endostatin, bevacizumab, genistein, interferon alpha, interleukin-2, interleukin-12, pazopanib, pegaptanib, ranibizumab, rapamycin, thalidomide; and growth inhibitory polypeptides such as erythropoietin, interleukins (e.g., IL-1, IL-2, IL-3, IL-6), leukemia inhibitory factor, interferons, thrombopoietin, TNF-α, CD30 ligand, 4-1BB ligand, and Apo-1 ligand. Also included are pharmaceutically acceptable salts, acids, or derivatives of any of the above listed agents.

In still other embodiments, the compositions may further comprise at least one anti-inflammatory agent. The anti-inflammatory agent may be a glucocorticoid steroid such as the naturally occurring hydrocortisone (cortisol), or synthetic glucocorticoids such as prednisone, prednisolone, methylprednisolone, dexamethasone, betamethasone, triamcinolone, beclometasone, fludrocortisones, deoxycorticosterone, alclometasone, fluocinonide, aldosterone, and derivatives thereof. Alternatively, the anti-inflammatory agent may be a non-steroidal anti-inflammatory agent (NSAID). Non-limiting examples of suitable NSAIDs include acetylsalicylic acid (aspirin), celecoxib, choline magnesium salicylate, Cox-2 inhibitors, diclofenac, diflunisal, etodolac, fenoprofen, flufenisal, flurbiprofen, ibuprofen, indomethacin, ketoprofen, ketorolac, meclofenamate, mefenamate, nabumetone, naproxen, oxaprozin, phenylbutazone, piroxicam, salsalate, sulindac, tolmetin, valdecoxib, and zomepirac.

The amount of the additional API included in the composition can and will vary depending upon the identity of the additional API.

(d) Dosage Forms

The compositions disclosed herein can be formulated into various dosage forms and administered by a number of different means that will deliver a therapeutically effective amount of the compound of Formula (I). Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980).

Solid dosage forms for oral administration include capsules, tablets, geltabs, caplets, gelcaps, pills, powders, pellets, and granules. In such solid dosage forms, the active ingredient is ordinarily combined with one or more pharmaceutically acceptable excipient, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil.

For topical (e.g., transdermal or transmucosal) administration, penetrants appropriate to the barrier to be permeated are generally included in the preparation. Transmucosal administration may be accomplished through the use of nasal sprays, aerosol sprays, tablets, or suppositories, and transdermal administration may be via ointments, salves, gels, patches, or creams as generally known in the art.

### (III) Methods for Using Compounds of Formula (I)

Yet another aspect of the present disclosure provides methods for using the compounds of Formula (I) to inhibit activation of DDR2 and/or inhibiting cell migration in cells that express DDR2. Also provided are methods for treating a disease or disorder associated with DDR2 dysfunction.

(a) Method for Inhibiting Activation of DDR2

The method comprises contacting DDR2 with an effective amount of a compound of Formula (I) such that activation of DDR2 is inhibited. The compound of Formula (I):

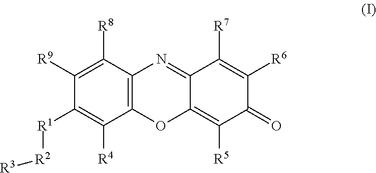

wherein R¹, R⁴, R⁵, R⁶, R⁷, R⁸, and R⁹ are as defined above in Section (I), R² is alkyl, substituted alkyl, carbonyl, ester, amide, sulfinyl, sulfinyl, or a bond, and R³ is hydrogen, hydrocarbyl, or substituted hydrocarbyl.

In some embodiments, R² may be C₁-C₅ alkyl or C₁-C₅ substituted alkyl. For example, R² may be —CH₂— (methylene), —(CH₂)₂—, —(CH₂)₃—, F—(CH₂)₄—, or —(CH₂)₅—. Alternatively, R² may be an alkyl comprising one or more alkyl, amino, hydroxyl, keto, nitro, phospho, or thiol substituents. In other embodiments, R² may be carbonyl (—C(O)—), ester (—C(O)O—), amide (—C(O)NH—), sulfinyl (—S(O)—), or sulfonyl (—S(O)₂—). In still other embodiments, R² may be absent (and R¹ is linked to R³).

In various embodiments, R³ may be hydrogen, alkyl, substituted alkyl, alkenyl, substituted alkenyl, alkynyl, substituted alkynyl, heterocyclic, substituted heterocyclic, aryl, or substituted aryl. In various embodiments, R³ may be alkyl, substituted alkyl, phenyl, substituted phenyl, naphthyl, substituted naphthyl, furyl, substituted furyl, benzofuryl, substituted benzofuryl, oxazolyl, substituted oxazolyl, isoxazolyl, substituted isoxazolyl, oxadiazolyl, substituted oxadiazolyl, benzoxazolyl, substituted benzoxazolyl, benzoxadiazolyl, substituted benzoxadiazolyl, pyrrolyl, substituted pyrrolyl, pyrazolyl, substituted pyrazolyl, imidazolyl, substituted imidazolyl, triazolyl, substituted triazolyl, tetrazolyl, substituted tetrazolyl, pyridyl, substituted pyridyl, pyrimidyl, substituted pyrimidyl, pyrazinyl, substituted pyrazinyl, pyridazinyl, substituted pyridazinyl, piperidyl, substituted piperidyl, indolyl, substituted indolyl, isoindolyl, substituted isoindolyl, indolizinyl, substituted indolizinyl, morpholyl, substituted morpholyl, benzimidazolyl, substituted benzimidazolyl, indazolyl, substituted indazolyl, benzotriazolyl, substituted benzotriazolyl, tetrazolopyridazinyl, substituted tetrazolopyridazinyl, carbazolyl, substituted carbazolyl, purinyl, substituted purinyl, quinolinyl, substituted quinolinyl, isoquinolinyl, substituted isoquinolinyl, imidazopyridyl, or substituted imidazopyridyl. In specific embodiments, R³ may be phenyl, substituted phenyl (e.g., phenylamine, hydroxyphenyl, methoxyphenyl, dihalomethoxyphenyl, benzoate, methyl benzoate, ethyl benzoate, benyzl, substituted benzyl, phenylsulfinyl, phenylsulfonyl, etc.), pyridyl, substituted pyridyl, furyl, or substituted furyl. In certain embodiments, R³ may be sulfonyl substituted phenyl (e.g., N-methylbenzenesulfonamide, N-ethylbenzenesulfonamide, or phenylsulfonylmorpholine).

In specific embodiments, R¹ is O or NH, R² is methylene, carbonyl, amide, or sulfonyl, R³ is phenyl, substituted phenyl, pyridyl, substituted pyridyl, furyl, or substituted furyl, and R⁴-R⁹ are hydrogen.

The compounds of Formula (I) generally inhibit the binding of DDR2 to its ligand (i.e., fibrillar collagens and the non-fibrillar collagen type X) in a non-competitive manner (see Example 2 below). Thus, the compounds of Formula (I) do not interact with the collagen-binding pocket in the discoidin (DS) domain of DDR2. Rather, the compounds of Formula (I) bind to an allosteric site on DDR2. DDR2 may be a naturally occurring protein or DDR2 may be a recombinantly expressed protein. DDR2 may be wild type or mutant. In some embodiments, DDR2 may be within a cell. The cell may be a mammalian cell. The mammalian cell may be a human cell or a non-human animal cell. Cells known to express DDR2 include tumor cells, metastatic cancer cells, synovial fibroblasts, skin fibroblasts, chondrocytes, neutrophils, dendritic cells, hepatic stellate cells, vascular smooth muscle cells, adipocytes, and osteoblasts. In other embodiments, DDR2 may be isolated from a cell.

The amount of the compound of Formula (I) that is used in the method can and will vary depending, for example, upon the identity of the compound of Formula (I). Persons of skill in the art are familiar with means for determining the appropriate amount to use in the method.

In general, the compound of Formula (I) inhibits activation of DDR2 by at least about 5%. In various embodiments, the compound of Formula (I) may inhibit DDR2 activation of by at least about 10%, at least about 30%, at least about 50%, at least about 100%, at least about 200%, or at least about 500%.

(b) Method for Inhibiting Cell Migration

The method comprises contacting a cell that expresses DDR2 with an effective amount of a compound of Formula (I) such that cell migration, cell invasion, and/or cell adhesion is inhibited. The compound of Formula (I):

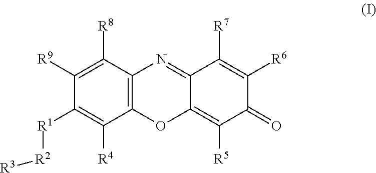

wherein R¹, R², R³, R⁴, R⁵, R⁶, R⁷, R⁸, and R⁹ are as defined above in section (II).

In some embodiments, the cell may be mammalian cell. The mammalian cell may be a human cell or a non-human animal cell. The cell may be a primary cell or a tissue culture cell. Cells known to express DDR2 include tumor cells, metastatic cancer cells, synovial fibroblasts, skin fibroblasts, cancer associated fibroblasts, chondrocytes, neutrophils, dendritic cells, hepatic stellate cells, vascular smooth muscle cells, adipocytes, endothelial cells, and osteoblasts. In certain embodiments, the cell may be a human or mouse tumor cell line cell such as BT549, 4T1, MDA-MB-23, HEK293, HEK293T, HELA, W138, Hep G2, U2-OS, A-431, A549, or K562 cells.

In various embodiments, the cell may be in vitro. In other embodiments the cell may be in situ, i.e., in a subject of interest. In general, the subject will be a mammal. In some embodiments, the subject may be a human. In other embodiments, the subject may be a non-human animal. Non-limiting examples of non-human animals include research animals (e.g., mice, rats, rabbits, primates), companion animals (e.g., cats, dogs, horses, rabbits, gerbils), agricultural animals (e.g., cows, pigs, sheep, goats, fowl), and zoo animals (e.g., lions, tiger, elephants, and the like).

The amount of the compound of Formula (I) that is used in the method can and will vary depending, for example, upon the identity of the compound of Formula (I) and the cell used in the method. Persons skilled in the art are familiar with means for determining the appropriate amount to use in the method.

In general, the compound of Formula (I) inhibits cell migration (or cell invasion or cell adhesion) by at least about 5%. In various embodiments, the compound of Formula (I) may inhibit cell migration (or cell invasion or cell adhesion) by at least about 10%, at least about 30%, at least about 50%, at least about 100%, at least about 200%, or at least about 500%.

(c) Method for Treating a Disease or Disorder Associated with DDR2 Dysfunction

The method comprises administering to a subject in need thereof an effective amount of a compound of Formula (I) such that the symptoms and/or progression of the disease or disorder are alleviated and/or inhibited. The compound of Formula (I):

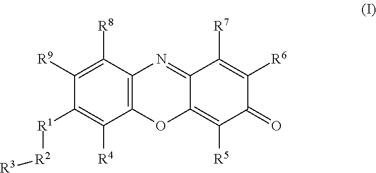

wherein R¹, R², R³, R⁴, R⁵, R⁶, R⁷, R⁸, and R⁹ are as defined above in section (II).

In general, the disease or disorder associated with DDR2 dysfunction is a cancer or as inflammatory disorder.

Non-limiting examples of cancers that may be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood). In specific embodiments, the cancer to be treated may be breast cancer, ovarian cancer, lung cancer, head and neck cancer, liver cancer, or colon cancer. Administration of the compound of Formula (I) may prevent or reduce metastasis of cancer cells, inhibit or slow the growth of cancer cells, and/or reduce or eliminate tumors.

Inflammatory disorders that can be treated include acute and chronic inflammatory disorders. For example, the inflammatory disorder may be arthritis including, but not limited to, rheumatoid arthritis, osteoarthritis, spondyloarthropathies, gouty arthritis, systemic lupus erythematosus, or juvenile arthritis. In other embodiments, the inflammatory disorder may be a fibrosis, include without limit, pulmonary fibrosis, cystic fibrosis, liver fibrosis (i.e., cirrhosis), renal fibrosis, skin fibrosis, scleroderma, atrial fibrosis, endomyocardial fibrosis, arthrofibrosis, intestinal fibrosis (i.e., Crohn's disease), or myelofibrosis. In another embodiment, the inflammatory disorder may be associated wound healing. In further embodiments, the inflammatory disorder may be associated with asthma, allergic rhinitis, sinus diseases, bronchitis, tuberculosis, acute pancreatitis, sepsis, infectious diseases, menstrual cramps, premature labor, tendinitis, bursitis, skin-related conditions such as psoriasis, eczema, atopic dermatitis, urticaria, dermatitis, contact dermatitis, and burns, or from post-operative inflammation including from ophthalmic surgery such as cataract surgery and refractive surgery. In a further embodiment, the inflammatory disorder may be a gastrointestinal condition such as inflammatory bowel disease Crohn's disease, gastritis, irritable bowel syndrome, chronic cholecystitis, or ulcerative colitis. In yet another embodiment, the inflammation may be associated with diseases such as vascular diseases, migraine headaches, periarteritis nodosa, thyroiditis, aplastic anemia, Hodgkin's disease, sclerodoma, rheumatic fever, type I diabetes, autoimmune diseases, neuromuscular junction diseases including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, allergic rhinitis, respiratory distress syndrome, systemic inflammatory response syndrome (SIRS), cancer-associated inflammation, reduction of tumor-associated angiogenesis, endotoxin shock syndrome, atherosclerosis, and the like. In an alternate embodiment, the inflammatory disorder may be associated with an ophthalmic disease, such as retinitis, retinopathies, uveitis, ocular photophobia, or of acute injury to the eye tissue. In still another embodiment, the inflammation may be a pulmonary inflammation, such as that associated with viral infections or cystic fibrosis, chronic obstructive pulmonary disease, or acute respiratory distress syndrome. The inflammatory disorder may also be associated with tissue rejection, graft v. host diseases, delayed-type hypersensitivity, as well as immune-mediated and inflammatory elements of neurological diseases such as Alzheimer's, Parkinson's, multiple sclerosis, and the like. In specific embodiments, the inflammatory disorder to be treated may be rheumatoid arthritis, osteoarthritis, pulmonary fibrosis, cirrhosis, renal fibrosis, or skin fibrosis. Administration of the compound of Formula (I) may reduce or eliminate the pain, stiffness, and/or swelling associated with the inflammatory disorder.

The compound of Formula (I) may be administered orally (as a solid or a liquid), parenterally (which includes intramuscular, intravenous, intradermal, intraperitoneal, and subcutaneous), or topically (which includes transmucosal and transdermal). An “effective” amount refers to the dose of the compound that provides beneficial effects to the subject. The amount to be used can be determined by the skilled practitioner in view of desired dosages and side effects of the compound. The frequency of administration can and will vary depending, for example, on the pharmacokinetics of the compounds and disease or disorder to be treated.

In some embodiments, the method further comprises co-administration of an orthosteric DDR2 inhibitor, a receptor tyrosine kinase inhibitor, a chemotherapeutic agent, and/or an anti-inflammatory agent, examples of which are described above in section (II). In some embodiments, the compound of Formula (I) and the additional API may be part of the same composition (see section (II) above). In other embodiments, each of the compound of Formula (I) and the additional API may be a separate composition. The separate compositions may be administered concurrently or sequentially.

In general, the subject is a mammal. The subject can be male or female, young or old. In some embodiments, the subject may be a human. In other embodiments, the subject may be a non-human animal. Non-limiting examples of non-human animals include research animals (e.g., mice, rats, rabbits, primates), companion animals (e.g., cats, dogs, horses, rabbits, gerbils), agricultural animals (e.g., cows, pigs, sheep, goats, fowl), and zoo animals (e.g., lions, tiger, elephants, and the like).

### Definitions

Methods and compositions described herein utilize laboratory techniques well-known to skilled artisans. Such technique guidance can be found in laboratory manuals and textbooks such as Hedrickson et al., Organic Chemistry 3rd edition, McGraw Hill, New York, 1970; Carruthers, W., and Coldham, I., Modern Methods of Organic Synthesis (4th Edition), Cambridge University Press, Cambridge, U.K., 2004, Li, W., et al., Fischbach, F., and Dunning, M. B., A Manual of Laboratory and Diagnostic Tests, Lippincott Williams & Wilkins Philadelphia, Pa., 2004; Sambrook, J., et al., Molecular Cloning: A Laboratory Manual, 3rd ed. Cold Spring Harbor Laboratory Press, Cold Spring Harbor, N.Y., 2001. Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003.

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “acyl,” as used herein alone or as part of another group, denotes the moiety formed by removal of the hydroxyl group from the group COOH of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo, and R² is hydrogen, hydrocarbyl, or substituted hydrocarbyl.

The term “acyloxy,” as used herein alone or as part of another group, denotes an acyl group as described above bonded through an oxygen linkage (O), e.g., RC(O)O— wherein R is as defined in connection with the term “acyl.”

As used herein, the term “aliphatic” refers to a hydrocarbyl group in which the carbon atoms are linked in open chains, i.e., either linear or branched but not cyclic. Alkyl, alkenyl, and alkynyl groups, optionally substituted, are aliphatic.

The term “alkyl” as used herein describes groups containing from one to thirty carbon atoms in the principal chain. They may be straight or branched chain or cyclic and include methyl, ethyl, propyl, isopropyl, butyl, hexyl and the like.

The term “alkenyl” as used herein describes groups containing from two to thirty carbon atoms in the principal chain and further comprising at least one carbon-carbon double bond. They may be straight or branched chain or cyclic and include ethenyl, propenyl, isopropenyl, butenyl, isobutenyl, hexenyl, and the like.

The term “alkoxide” or “alkoxy” as used herein is the conjugate base of an alcohol. The alcohol may be straight chain, branched, cyclic, and includes aryloxy compounds.

The term “alkynyl” as used herein describes groups containing from two to thirty carbon atoms in the principal chain and further comprising at least one carbon-carbon triple bond. They may be straight or branched chain and include ethynyl, propynyl, butynyl, isobutynyl, hexynyl, and the like.

The term “amide” as used herein describes a compound comprising a carbonyl-nitrogen linkage.

The term “aminoacyl” refers to an amino acid residue.

The term “aromatic” as used herein alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The term “aryl” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “halogen” or “halo” as used herein alone or as part of another group refer to chlorine, bromine, fluorine, and iodine.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, piperidyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. They may be straight, branched, or cyclic.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

The term “treating,” as used herein, refers to inhibiting or alleviating the symptoms of the disease or disorder; reversing, inhibiting, or slowing the progression of the disease or disorder; and/or preventing or delaying the onset of the disease or disorder. The term “treatment”, as used herein, unless otherwise indicated, refers to the act of treating as “treating” is defined immediately above.

Having described the invention in detail, it will be apparent that modifications and variations are possible without departing from the scope of the invention defined in the appended claims.

### EXAMPLES

The following examples illustrate certain aspects of the invention.

### Example 1: Synthesis of DDR2 Inhibitors

Derivatives of 7-hydroxy-phenoxazine-3-one were synthesized using standard procedures according to the following scheme:

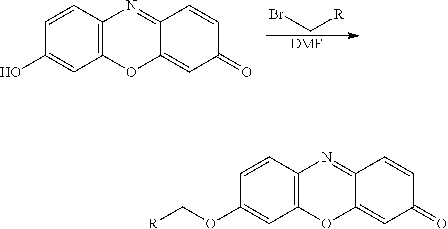

Examples of the R-containing compound that were used are shown below. The compounds were characterized by mass spectrophotometry and nuclear magnetic resonance spectrometry.

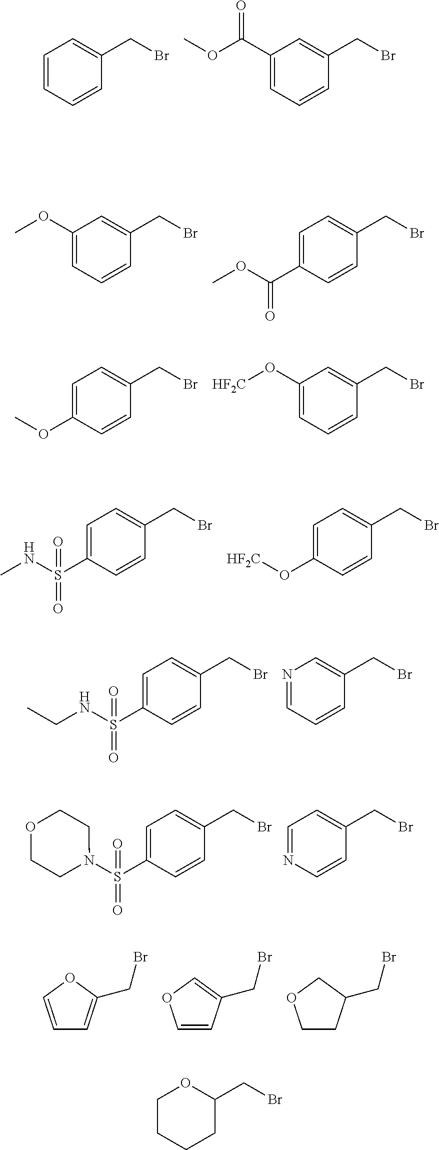

The scheme below illustrates methods for preparing intermediate compounds with different linker (i.e., R²) groups.

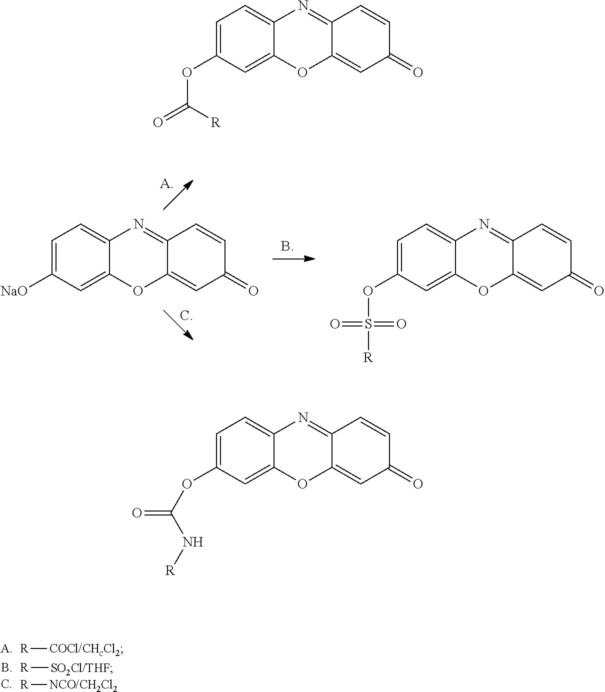

Methods for preparing compounds in which R¹ is NH are diagrammed below.

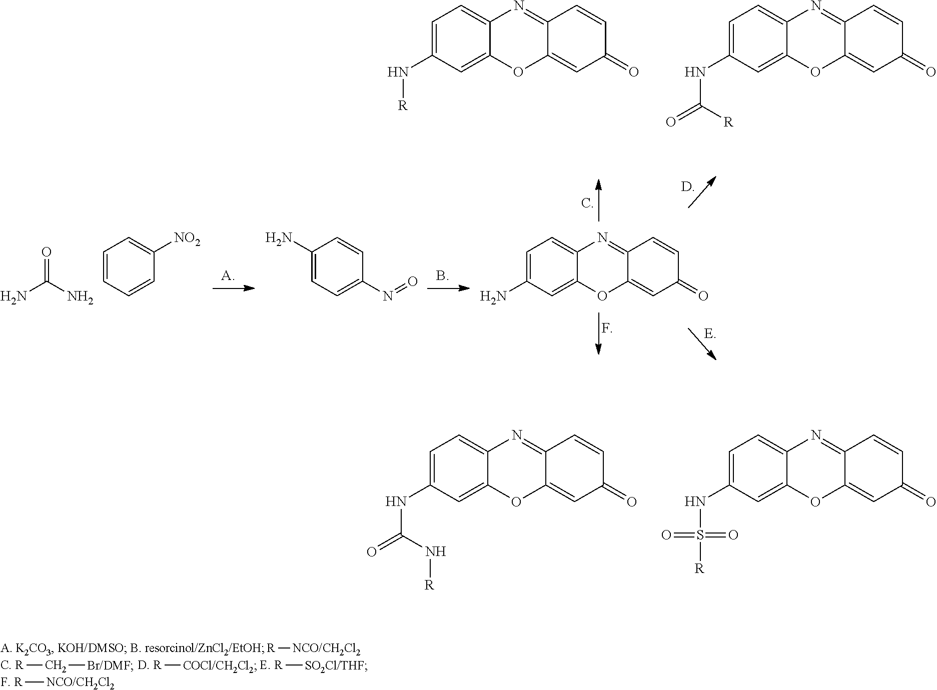

### Example 2: In Vitro Studies with DDR2 Inhibitors

The activity of the synthesized compounds was assayed using an in vitro ELISA-type assay to measure the binding of purified, soluble DDR2 extracellular domain (ECD) to an immobilized substrate. The substrate used was a high affinity, specific, triple helical peptide (see Xu et al., PLoS One. 2012; 7(12): e52209), which represents the minimal DDR2 specific binding domain within collagens I-III.

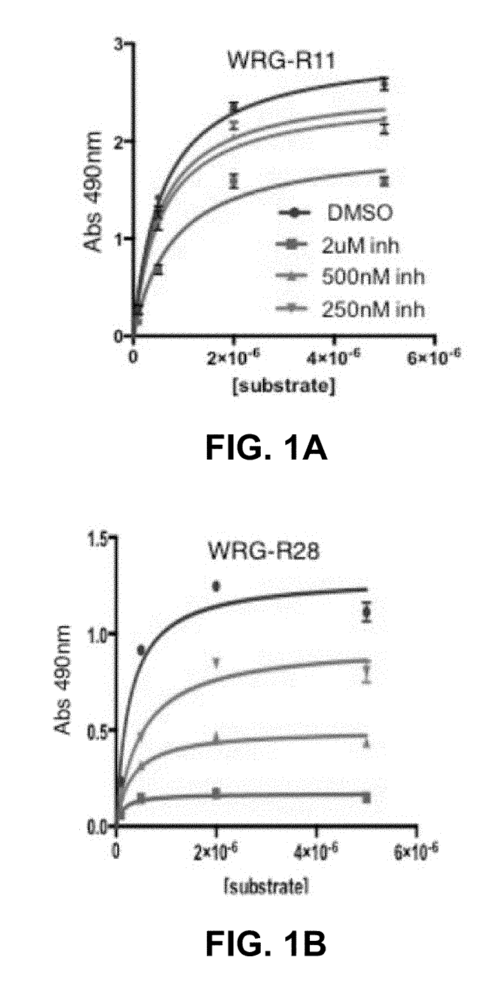

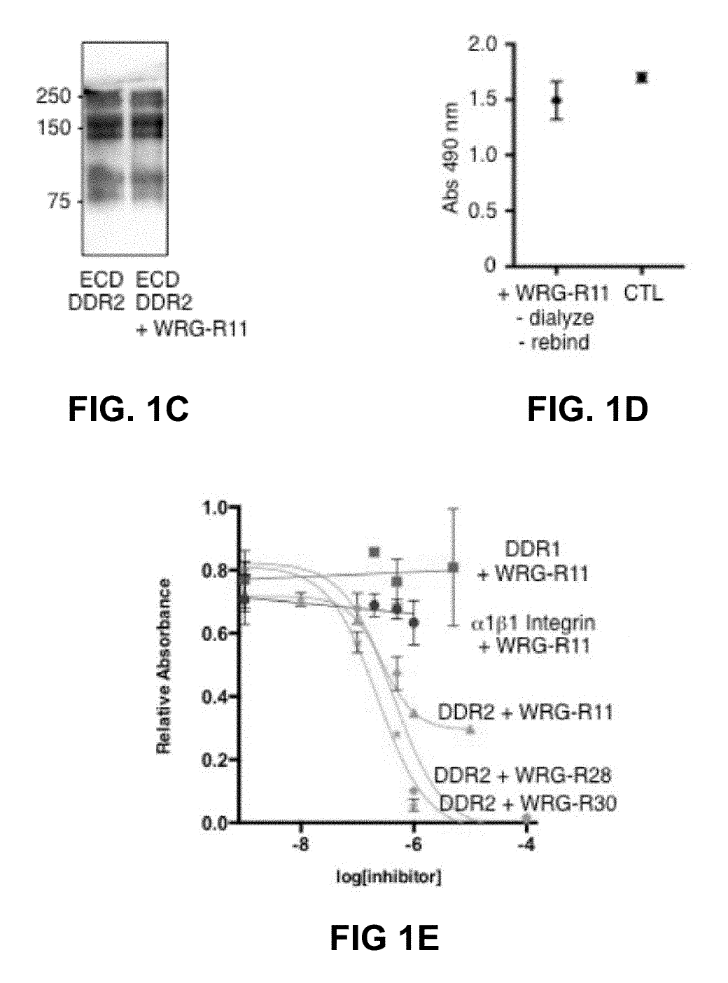

DDR2-collagen binding activity was measured in the presence of 250 nM, 500 nM, or 2 μM of test compound or DMSO control. As shown in FIG. 1A and FIG. 1B, WRG-11 and WRG-28, respectively, decreased DDR2-collagen binding activity, even at the lowest dose tested (250 nM). The data indicate that compounds WRG-R11 and WRG-R28 act in a non-competitive manner to inhibit DDR2 binding to collagen. FIG. 1C, shows that WRG-R11 does not disrupt DDR2 ECD dimer formation. (PAGE under non-reducing conditions in the absence or presence of 1 μM WRG-R11 is shown. Dimers of DDR2 run at 150 kDa.) FIG. 1D shows that WRG-R11 does not covalently modify the extracellular domain (ECD) of DDR2 to irreversibly alter its ability to bind to collagen. The data further illustrate that the compounds are highly specific or selective, as they do not inhibit other collagen receptors (i.e., integrin or related RTK DDR1) from binding to collagen (see FIG. 1E). These data indicate that the compounds tested are not acting as traditional orthosteric inhibitors and that they can act as allosteric inhibitors.

Table 1 presents compounds that were tested and their IC₅₀ values.

TABLE 1
Inhibition of DDR2-collagen binding activity

| Compound | R¹ | R² | R³ | IC₅₀ |
|---|---|---|---|---|
| 7-hydroxy-phenoxazin-3- | O | — | H | 6 μM |
| one | | | | |
| 7-ethyloxy-phenoxazin-3- | O | CH₂ | CH₃ | 4 μM |
| one | | | | |
| 7-pentyloxy-phenoxazin- | O | CH₂ | (CH₂)₃CH₃ | 3.5 μM |
| 3-one (WRG-1) | | | | |
| 7-benzyloxy-phenoxazin- | O | CH₂ | C₆H₅ | 461 nM |
| 3-one (WRG-2) | | | | |
| WRG-R-3 | O | CH₂ | 4-methyl benzoate | 347 nM |
| | | | (C₆H₅COOCH₃) | |
| WRG-R-4 | O | CH₂CH₂ | OCH₃ | 14 μM |
| WRG-R-5 | O | CH₂ | 3-methoxy benzene | 393 nM |
| | | | (CH₃OC₆H₄) | |
| WRG-R-6 | O | CO | C₆H₅ | 1.41 μM |
| WRG-R-7 | O | SO₂ | C₆H₅ | 558 nM |
| WRG-R-9 | O | CH₂ | 3-pyridine (C₅H₄N) | 1.2 μM |
| WRG-R-10 | O | CH₂ | 4-pyridine (C₅H₄N) | 311 nM |
| WRG-R-11 | O | CH₂ | 4-methoxy benzene | 252 nM |
| | | | (CH₃OC₆H₄) | |
| WRG-R-12 | O | C(O)NH | C₆H₅ | >100 μM |
| WRG-R-13 | NH | C(O)NH | C₆H₅ | * |
| WRG-R-16 | NH | C(O)NH | 4-methoxy benzene | * |
| | | | (CH₃OC₆H₄) | |
| WRG-R-14 | NH | C(O) | C₆H₅ | * |
| WRG-R-15 | NH | C(O) | 4-methoxy benzene | * |
| | | | (CH₃OC₆H₄) | |
| WRG-R-20 | NH | S(O₂) | C₆H₅ | >100 μM |
| WRG-R-18 | NH | S(O₂) | 4-methoxy benzene | * |
| | | | (CH₃OC₆H₄) | |
| WRG-R-19 | O | CH₂ | 4-benzoic acid | * |
| | | | (C₆H₅COOH) | |
| WRG-R-27 | O | CH₂ | 4-difluoromethoxy benzene | 110 nM |
| | | | (CF₂HOC₆H₄) | |
| WRG-R-28 | O | CH₂ | N-ethyl-4-benzene | 230 nM |
| | | | sulfonamide | |
| WRG-R-30 | NH | S(O₂) | 4-benzene sulfonyl | 432 nM |
| | | | morpholine | |
| WRG-R-31 | O | CH₂ | N-methyl-4-benzene | * |
| | | | sulfonamide | |
| WRG-R-32 | O | CH₂ | 2-methyl furan | * |
| WRG-R-33 | O | CH₂ | 3-methyl furan | * |
| WRG-R-34 | O | CH₂ | 3-methyl tetrahydrofuran | * |
| WRG-R-35 | O | CH₂ | 2-methyl tetrahydropyran | * |
| WRG-R-36 | O | CH₂ | 3-methyl benzoate | * |
| WRG-R-37 | O | CH₂ | 3-difluoromethoxy benzene | * |

* Not determined

### Example 3: Cell-Based Studies

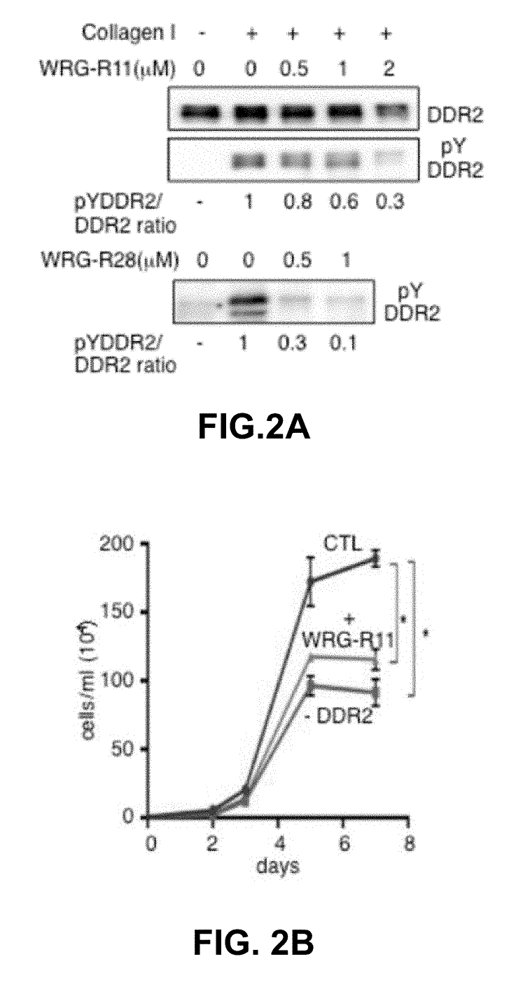

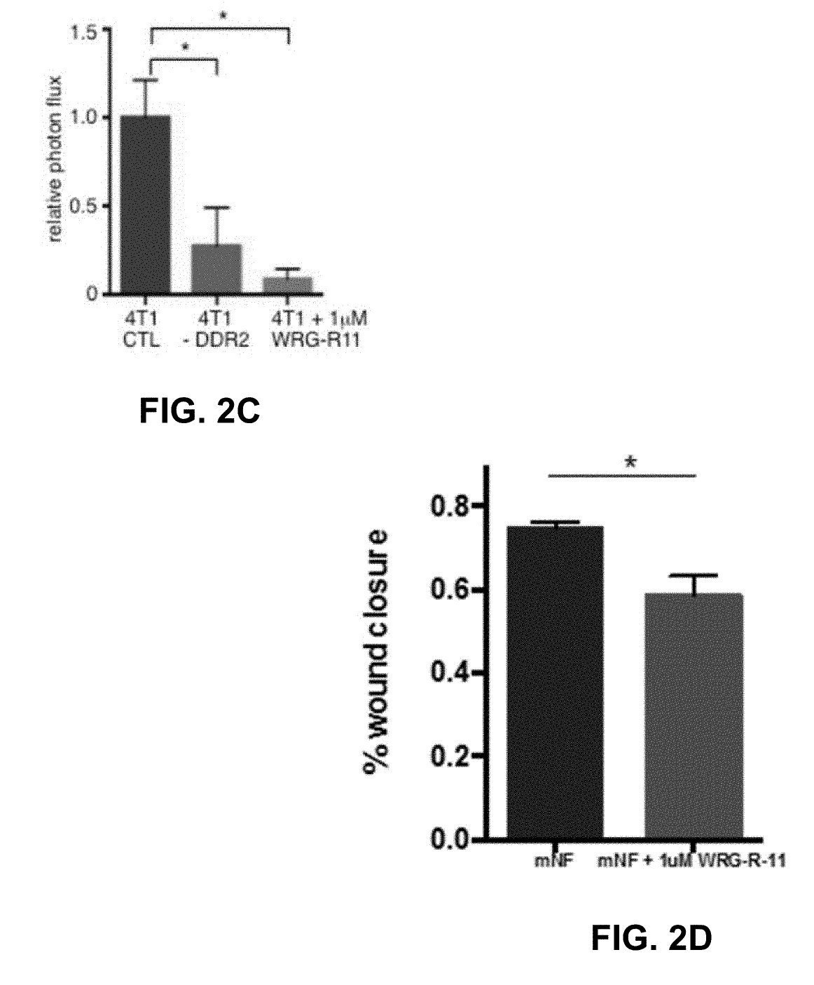

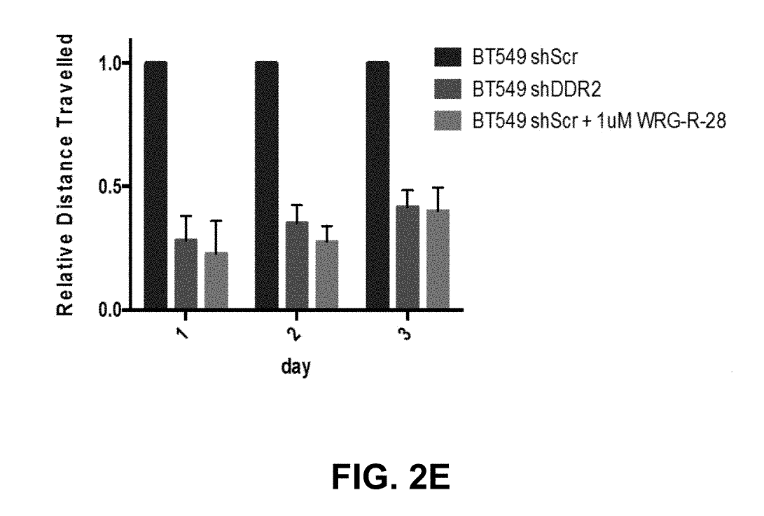

DDR2 expressing HEK293 cells were added to plates pated with collagen I and incubated for 8 h in the presence of increasing amounts of WRG-11 or WRG-28. Cells were lysed, DDR2 was immunoprecipitated, SDS-PAGE was performed, and Western blots with DDR2 Ab or anti-PY Ab were performed. The results are show in FIG. 2A. For each compound, the amount of pY-DDR2 relative to total DDR2 is quantified and listed below each image. The inhibitor compounds inhibited activation (i.e., phosphorylation) of DDR2.

4T1 breast cancer cells were treated with SCR RNAi (control), DDR2 RNAi (-DDR2), or SCR RNAi and 1 μM WRG-R-11 (+WRG-11) Cell proliferation over 7 days is plotted in FIG. 2B. Cell migration was analyzed using an in vitro gel matrix (MATRIGEL®, Corning) cell invasion assay. FIG. 2C presents the results, which are plotted as the number of cells invading through to lower transwell surface membrane relative to control as determined by bioluminescence at 48 hours. The data show that the inhibitor compounds inhibit cancer cell proliferation and migration.

In a scratch wound assay, WRG-R-11 decreased migration of mouse derived fibroblasts (see FIG. 2D). Fibroblasts are an important cell type within the tumor stromal microenvironment that help to promote metastasis, and fibroblasts are involved in many inflammatory disorders such as arthritis.

FIG. 2E illustrates that WRG-R-28 impairs breast cancer cell migration through a 3D collagen I matrix. BT549 human metastatic breast cancer cells were treated with SCR RNAi (shSCR), DDR2 RNAi (shDDR2), or SCR RNAi and 1 μM WRG-R-28 (shSCR+WRG-28) Results are plotted as distance of cell migration through 3D collagen I matrix relative to control over three days.

Cells that do not express DDR2 were exposed to several concentrations of WRG-28 or WGR-30 and no cell growth inhibition or cell toxicity was observed.

### Example 4: Inhibition of Downstream Phosphorylation

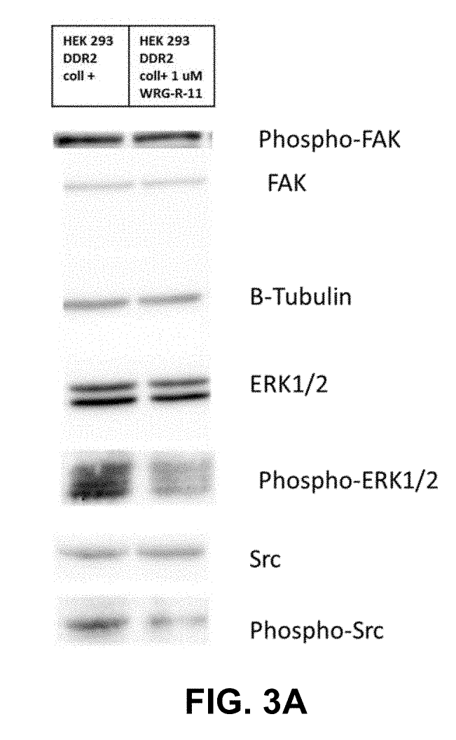

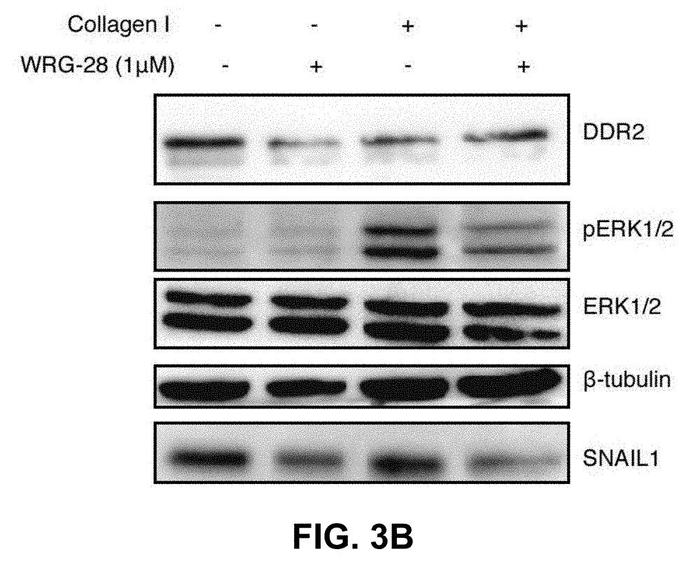

HEK293 cells expressing DDR2 were added to collagen I-coated plates and incubated for 6 hours in the absence or presence of 1 μM WRG-11 or WRG-28. Cells were lysed and Western blot analysis of DDR2 signaling was performed. As shown in FIG. 3A and FIG. 3B, the inhibitor compounds also inhibited downstream signaling, e.g., reduced levels of phospho-ERK1/2, phospho-SRC, and SNAIL1. Importantly, integrin activation in response to collagen stimulation is not impaired by this compound (i.e., no change in phosphor-FAK, see FIG. 3A), indicating its specificity in the cellular setting.

### Example 5: In Vivo Studies

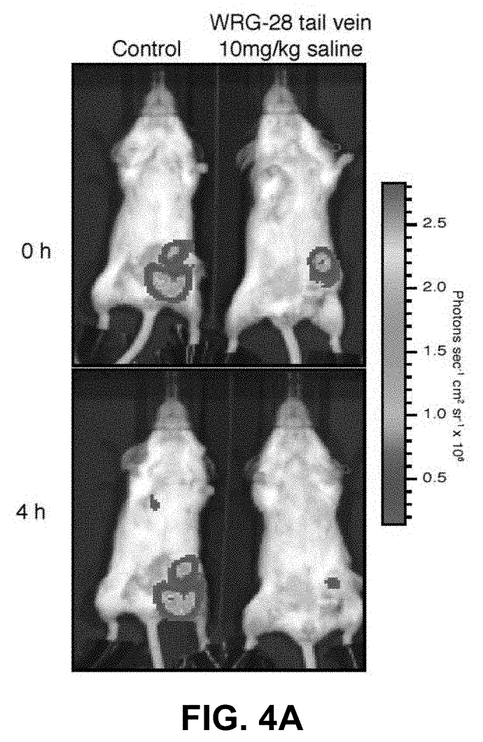

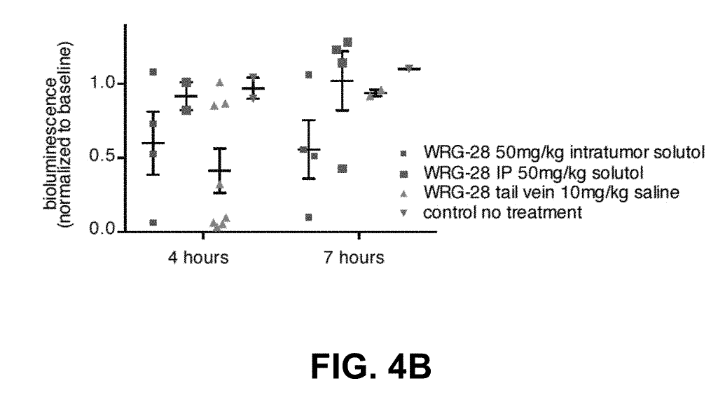

BALB/cJ mice whose breast tissue was implanted with 1×10⁶ 4 T1-Snail-CBG (clic beetle red) bioluminescent fusion protein were used for in vivo studies. Whole mouse bioluminescence imaging was performed at baseline and after administration of 10 mg/kg of WRG-28 or vehicle via tail vein injection. As shown in FIG. 4A, Snail-CBG bioluminescence signal (i.e. Snail stability) was decreased at 4 hours in mice treated with the inhibitor compound. FIG. 4B, presents the relative bioluminescent of 4T1-SNAIL-CBG tumor bearing mice relative to their pre-treatment baseline after intratumor, IP, or tail vein administration of WRG-28 or vehicle control.

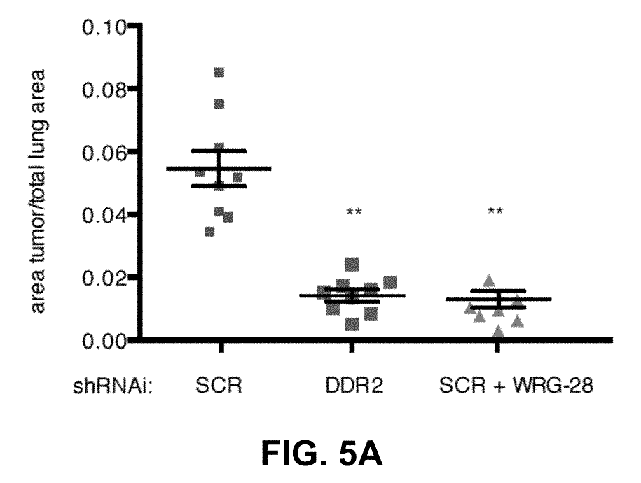

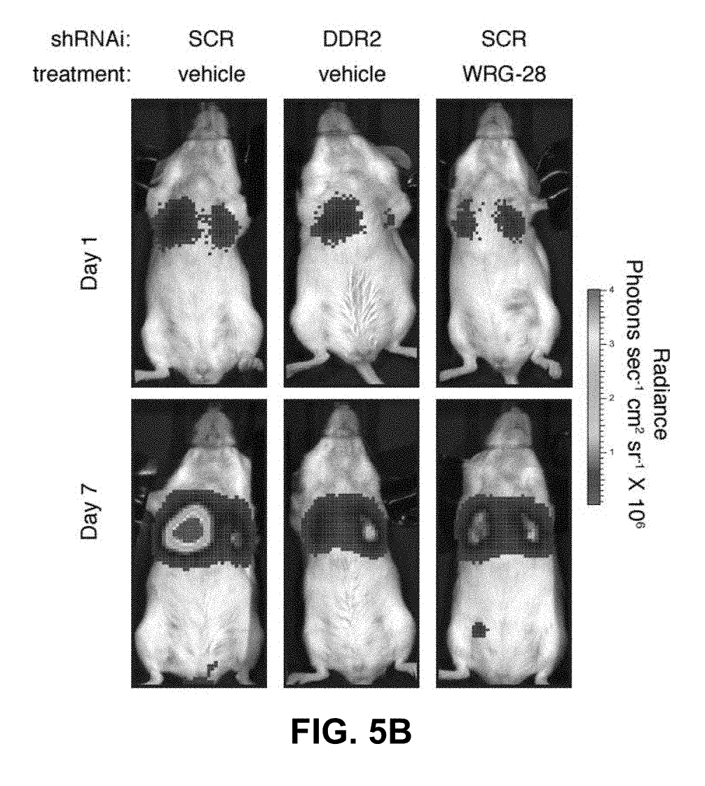

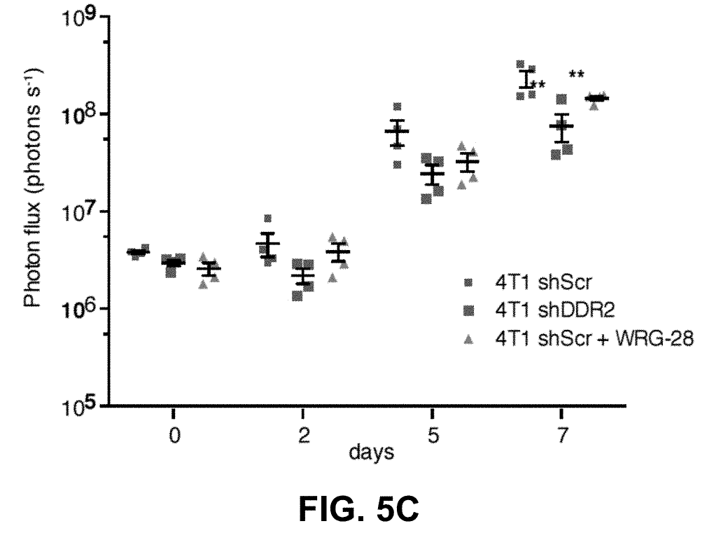

BALB/cJ mice were injected by tail vein 1×10⁵ 4 T1-GFP-luc expressing cells and lungs were evaluated for the presence of metastatic tumors in control (shSCR RNAi), DDR2 depleted (shDDR2 RNAi), and WRG-28 treated (shSCR RNAi+WRG-28) animals (four mice per condition). As shown in FIG. 5A, DDR2 depleted and WRG-28 treated mice had reduced tumor area per lung area. Whole mouse imaging at days 1 and 7 showed reduced bioluminescence in the DDR2 depleted and WRG-28 treated mice (FIG. 5B). FIG. 5C presents a quantification of the results.

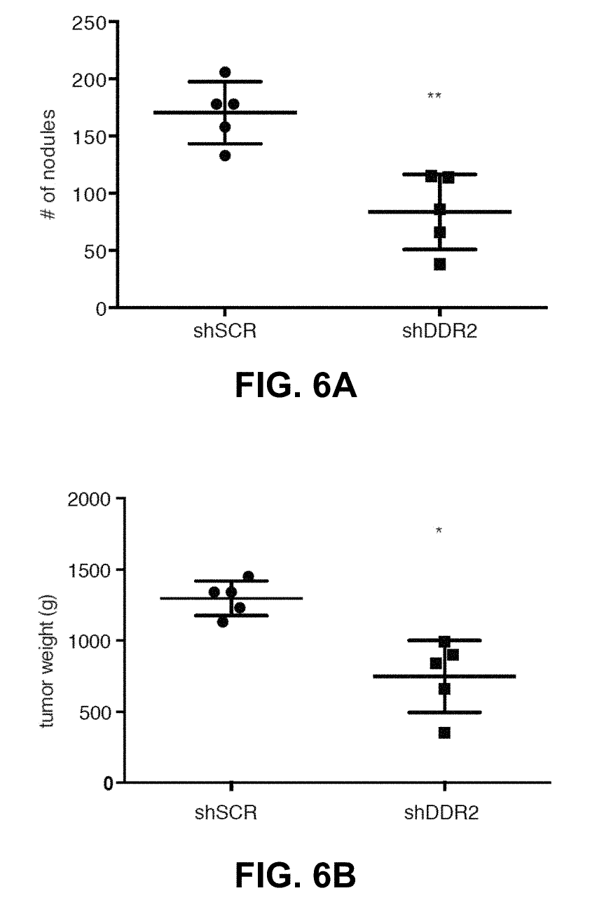

In a mouse model of ovarian cancer metastasis, examination of the peritoneal cavity from mice injected IP with either control (shSCR) and DDR2 depleted (shDDR2) A2780 human ovarian cancer cell lines revealed that the DDR2 depleted animals had fewer nodules and decreased tumor weights (FIG. 6A and FIG. 6B, respectively). Five mice per each experimental group were quantified.

1. A compound of Formula (I): 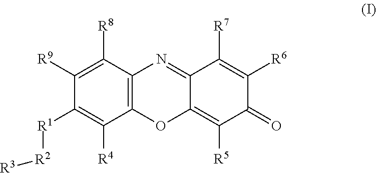
wherein:
R¹ is O or NH;
R² is alkyl, substituted alkyl, carbonyl, ester, amide, sulfinyl, or sulfonyl;
R³ is aryl, substituted aryl, heterocyclic, or substituted heterocyclic;
R⁴, R⁵, R⁶, R⁷, R⁸, and R⁹ independently are hydrogen, OR²⁰, NR²⁰R²¹, alkyl, substituted alkyl, halo, or thiol; and
R²⁰ and R²¹ independently are hydrogen, hydrocarbyl, or substituted hydrocarbyl;
provided that when R⁴-R⁹ are hydrogen, R¹ is O, and R² is methylene, then R³ is other than phenyl, hydroxyphenyl, hydrocarbyloxyphenyl, substituted hydrocarbyloxyphenyl, halophenyl, or haloalkylphenyl, or when R⁴-R⁹ are hydrogen, R¹ is O, and R² is carbonyl, then R³ is other than phenyl, hydroxyphenyl, methoxyphenyl, or furyl.

2. The compound of claim 1, wherein R³ is phenyl, substituted phenyl, naphthyl, substituted naphthyl, furyl, substituted furyl, benzofuryl, substituted benzofuryl, oxazolyl, substituted oxazolyl, isoxazolyl, substituted isoxazolyl, oxadiazolyl, substituted oxadiazolyl, benzoxazolyl, substituted benzoxazolyl, benzoxadiazolyl, substituted benzoxadiazolyl, pyrrolyl, substituted pyrrolyl, pyrazolyl, substituted pyrazolyl, imidazolyl, substituted imidazolyl, triazolyl, substituted triazolyl, tetrazolyl, substituted tetrazolyl, pyridyl, substituted pyridyl, pyrimidyl, substituted pyrimidyl, pyrazinyl, substituted pyrazinyl, pyridazinyl, substituted pyridazinyl, piperidyl, substituted piperidyl, indolyl, substituted indolyl, isoindolyl, substituted isoindolyl, indolizinyl, substituted indolizinyl, morpholyl, substituted morpholyl, benzimidazolyl, substituted benzimidazolyl, indazolyl, substituted indazolyl, benzotriazolyl, substituted benzotriazolyl, tetrazolopyridazinyl, substituted tetrazolopyridazinyl, carbazolyl, substituted carbazolyl, purinyl, substituted purinyl, quinolinyl, substituted quinolinyl, isoquinolinyl, substituted isoquinolinyl, imidazopyridyl, or substituted imidazopyridyl.

3. The compound of claim 1, wherein R⁴, R⁵, R⁶, R⁷, R⁸, and R⁹ independently are hydrogen, hydroxy, alkyoxy, amino, amine, alkyl, substituted alkyl, halo, or thio.

4. The compound of claim 1, wherein R² is methylene, carbonyl, amide, or sulfonyl, R³ is phenyl, substituted phenyl, pyridyl, substituted pyridyl, furyl, or substituted furyl, and R⁴-R⁹ are hydrogen.

5. The compound of claim 4, wherein R³ is sulfonyl substituted phenyl.

6. The compound of claim 1, wherein R¹ is O, R² is methylene, R³ is N-ethylbenzenesulfonamide, and R⁴-R⁹ are hydrogen

7. The compound of claim 1, wherein R¹ is O, R² is methylene, R³ is 4-(phenylsulfinyl)morpholine, and R⁴-R⁹ are hydrogen.

8. A composition comprising at least one compound of claim 1 and at least one pharmaceutically acceptable excipient.

9. The composition of claim 8, wherein the pharmaceutically acceptable excipient is chosen from a diluent, a binder, a filler, a buffering agent, a pH modifying agent, a disintegrant, a dispersant, a preservative, a lubricant, taste-masking agent, a flavoring agent, a coloring agent, or combination thereof.

10. The composition of claim 9, further comprising at least one additional pharmaceutically active ingredient (API).

11. The composition of claim 10, wherein the API is a Discoidin Domain Receptor 2 (DDR2) inhibitor, a receptor tyrosine kinase inhibitor, a chemotherapeutic agent, an anti-inflammatory agent, or combination thereof.

12. A method for inhibiting Discoidin Domain Receptor 2 (DDR2) activation, the method comprising contacting DDR2 with an effective amount of a compound of Formula (I): 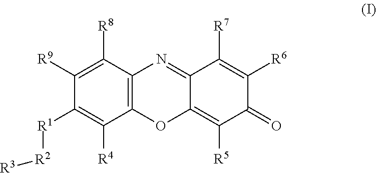
wherein:
R¹ is O or NH;
R² is alkyl, substituted alkyl, carbonyl, ester, amide, sulfinyl, sulfinyl, or is absent;
R³ is hydrogen, hydrocarbyl, or substituted hydrocarbyl;
R⁴, R⁵, R⁶, R⁷, R⁸, and R⁹ independently are hydrogen, OR²⁰, NR²⁰R²¹, alkyl, substituted alkyl, halo, or thiol; and
R²⁰ and R²¹ independently are hydrogen, hydrocarbyl, or substituted hydrocarbyl.

13. The method of claim 12, wherein R³ is hydrogen, alkyl, substituted alkyl, alkenyl, substituted alkenyl, alkynyl, substituted alkynyl, heterocyclic, substituted heterocyclic aryl, or substituted aryl.

14. (canceled)

15. The method of claim 12, wherein R⁴, R⁵, R⁶, R⁷, R⁸, and R⁹ independently are hydrogen, hydroxy, alkyoxy, amino, amine, alkyl, substituted alkyl, halo, or thio.

16. (canceled)

17. The method of claim 12, wherein the compound of Formula (I) binds to an allosteric site on DDR2.

18. The method of claim 12, wherein DDR2 is within a cell.

19.-20. (canceled)

21. A method for inhibiting cell migration, the method comprising contacting a cell that expresses Discoidin Domain Receptor 2 (DDR2) with an effective amount of a compound of Formula (I): 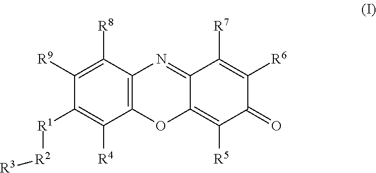
wherein:
R¹ is O or NH;
R² is alkyl, substituted alkyl, carbonyl, ester, amide, sulfinyl, sulfinyl, or is absent;
R³ is hydrogen, hydrocarbyl, or substituted hydrocarbyl;
R⁴, R⁵, R⁶, R⁷, R⁸, and R⁹ independently are hydrogen, OR²⁰, NR²⁰R²¹, alkyl, substituted alkyl, halo, or thiol; and
R²⁰ and R²¹ independently are hydrogen, hydrocarbyl, or substituted hydrocarbyl.

22. The method of claim 21, wherein R³ is hydrogen, alkyl, substituted alkyl, alkenyl, substituted alkenyl, alkynyl, substituted alkynyl, heterocyclic, substituted heterocyclic aryl, or substituted aryl.

23. (canceled)

24. The method of claim 21, wherein R⁴, R⁵, R⁶, R⁷, R⁸, and R⁹ independently are hydrogen, hydroxy, alkyoxy, amino, amine, alkyl, substituted alkyl, halo, or thio.

25. (canceled)

26. The method of claim 21, wherein the cell is a tumor cell, a synovial fibroblast, a skin fibroblast, a chondrocyte, a neutrophil, a dendritic cell, a hepatic stellate cell, or a vascular smooth muscle cell.

27.-41. (canceled)
